(19) United States
(12) Patent Application Publication
(10) Pub. No.: US 2025/0302907 A1
(43) Pub. Date: Oct. 2, 2025

(54) EXTERNALLY APPLIED MEDICATED WINE FOR TREATING CERVICAL SPONDYLOSIS OR LUMBAR SPONDYLOSIS CAUSED BY HYPEROSTOSIS AND APPLICATION THEREOF

(71) Applicant: Yongfang Wu, Kaili (CN)

(72) Inventor: Yongfang Wu, Kaili (CN)

(21) Appl. No.: 18/941,635

(22) Filed: Nov. 8, 2024

(30) Foreign Application Priority Data
Mar. 27, 2024 (CN) 2024103547752

(51) Int. Cl. A61K 36/8884; A61K 9/00; A61K 36/888; A61K 36/899; A61P 19/08

(52) U.S. Cl. CPC A61K 36/8884; A61K 9/0014; A61K 36/888; A61K 36/899; A61P 19/08

(57) ABSTRACT

An externally applied medicated wine for treating cervical spondylosis or lumbar spondylosis caused by hyperostosis and an application thereof, wherein the medicated wine is made of Arisaematis Rhizoma, Typhonium giganteum Engl., and baijiu (Chinese distilled spirits). With few types of drug ingredients, low cost, and no toxic side effects, the invention rationally adopts the compatibility of Arisaematis Rhizoma and Typhonium giganteum Engl., and takes baijiu as a guide drug, which can achieve the effects of dissipating masses, resolving swelling, promoting blood circulation, removing blood stasis, activating collaterals and quickly relieving pain. It can effectively treat cervical spondylosis and lumbar spondylosis caused by hyperotosis, and hyperostosis.

### 1. TECHNICAL FIELD

The invention belongs to the technical field of medicines, in particular to an externally applied medicated wine for treating cervical spondylosis or lumbar spondylosis caused by hyperostosis and an application thereof.

### 2. BACKGROUND ART

Osteoarthritis is the most common joint disease characterized by articular cartilage injuries and hyperostosis of surrounding bones and involving the entire periarticular tissues. As a degenerative change in human tissues, it may cause articular cartilage degeneration, fractures, defects, and even damage to the entire joint surface, manifesting as joint pain, stiffness, hypertrophy, and limited mobility. It is prone to occur in weight-bearing joints such as the knees, hips, cervical vertebrae, and lumbar vertebrae, usually persistent, stubborn, and difficult to stop or reverse. When hyperostosis is located in the lumbar vertebrae, it may cause lumbar spondylosis if not treated in time. When located in the cervical vertebrae, it may cause cervical spondylosis. Current treatments include physical therapy and surgical therapy. Physical therapy can only achieve local treatment but cannot make overall adjustments, and its effect is not ideal. Surgical therapy is not accepted by some patients due to the trauma. Drug therapy is also used, for example, taking non-steroidal anti-inflammatory drugs, but oral medications have great toxic side effects, which are not suitable for long-term use. Traditional Chinese medicine with less toxic side effects is also used for treatment, but some traditional Chinese medicine formulae are complex, with unknown drug actions. Therefore, there is an urgent need for drugs with definite drug actions, less toxic side effects, and suitable for long-term use.

### 3. SUMMARY OF THE INVENTION

Based on the above background, the invention provides an externally applied medicated wine for treating cervical spondylosis or lumbar spondylosis caused by hyperostosis and an application thereof, which has a simple composition, low cost, and significant drug actions.

The invention has the following technical solutions:

An externally applied medicated wine for treating cervical spondylosis or lumbar spondylosis caused by hyperostosis, wherein it is made of the following raw materials in parts by weight:
10-15 parts of Arisaematis Rhizoma, 3-5.5 parts of Typhonium giganteum Engl., and 12-18 parts of baijiu (Chinese distilled spirits).

Further, it is made of the following raw materials in parts by weight:
10-15 parts of Arisaematis Rhizoma, 3-5 parts of Typhonium giganteum Engl., and 12-18 parts of baijiu.

Further, it is made of the following raw materials in parts by weight:
15 parts of Arisaematis Rhizoma, 5.5 parts of Typhonium giganteum Engl., and 18 parts of baijiu.

Further, it is made of the following raw materials in parts by weight:
10 parts of Arisaematis Rhizoma, 3 parts of Typhonium giganteum Engl., and 12 parts of baijiu.

Further, the Arisaematis Rhizoma is Arisaematis Rhizoma Recens, and the Typhonium giganteum Engl. is Typhonium giganteum Engl. Recens.

Further, the baijiu has an ABV above 50%.

Based on the same invention conception, the invention applies the externally applied medicated wine for treating cervical spondylosis or lumbar spondylosis caused by hyperostosis to the preparation of medications for hyperostosis.

Though with few types of drug ingredients, low cost, and no toxic side effects when externally applied, the invention has been proved in clinical practice to have remarkable curative effect on cervical spondylosis and lumbar spondylosis caused by hyperostosis, and hyperostosis, wherein Arisaematis Rhizoma is bitter, dry, pungent, dispersing, warm, and toxic, and has strong potency, lung, liver and spleen tropism. It can be taken internally to eliminate dampness and phlegm, treat stubborn phlegm and cough, dispel pathogenic wind, stop spasms, and treat wind-phlegm patterns and tetanus; suitable for both damp phlegm and wind phlegm, particularly good for those with cold syndrome. Those with heat syndrome should pair the medicine with bitter and cold ingredients. Arisaematis Rhizoma can be used externally to attack toxins, resolve masses, reduce swelling, relieve pain, and treat abscesses and tuberculosis, especially good at removing wind phlegm from the meridians and stopping spasms. Typhonium giganteum Engl. can eliminate dampness and phlegm, dispel pathogenic wind, stop spasms, detoxify and resolve masses; when externally applied with baijiu, it can promote blood circulation, remove blood stasis, relax tendons, and activate collaterals. Acting as guide medicine, baijiu promotes drug absorption and may cause local irritation reactions to generate a stronger stimulation and local therapeutic effect of the drug on the surface of the site of action. The invention rationally adopts the compatibility of Arisaematis Rhizoma and Typhonium giganteum Engl., and takes baijiu as a guide drug, which can achieve the effects of dissipating masses, resolving swelling, promoting blood circulation, removing blood stasis, activating collaterals and quickly relieving pain. It can effectively treat cervical spondylosis and lumbar spondylosis caused by hyperostosis, and hyperostosis.

The invention can be used for the treatment of early and intermediate cancers such as breast cancer and skin cancer, with more obvious therapeutic effects and analgesic effects on the above cancer pain.

### 4. SPECIFIC EMBODIMENT OF THE INVENTION

To make the objectives, technical solutions, and advantages of the invention clearer, a further description of the invention will be given below in detail, in combination with the embodiments of the invention. It should be understood that the embodiments described herein are only to explain the invention, but not limiting.

### Typical Case

Case 1: Mr. Wu, male, 56 years old, felt numbness in his arms and dizziness in 2016. When he turned his head, his neck rattled, and was in pain. In 2017, the dizziness became more serious. He felt dizzy when he got out of bed every morning and even fell to the ground. After an X-ray examination at the hospital, it was found that hyperostosis occurred in the 3rd, 4th, and 5th cervical vertebrae, and he was diagnosed with cervical spondylosis. First, dry Arisaematis Rhizoma and Typhonium giganteum Engl. were used to soak in wine, which was ineffective. After four weeks of treatment with the medicated wine prepared in Embodiment 2, the numbness in his arms completely disappeared, and there was no pain in the neck. His head was not dizzy and did not rattle when turning, and it turned easily. After an X-ray examination, the hyperostosis in the 3rd, 4th, and 5th cervical vertebrae had basically disappeared. It has been 6 years since then, and there has been no recurrence. He works and lives normally.

Case 2: Ms. Li, female, 53 years old, was diagnosed with lumbar spondylosis in 2018, with discomfort and pain in the waist. Later, she was treated with Embodiment 2 of the invention. After about one month of treatment, the lumbar hyperostosis disappeared, and the waist movement was restored. There was no pain even when playing badminton. It has been five years since then, and there has been no abnormal reaction.

The above is only the preferred embodiment of the invention. It should be noted that various modifications and improvements based on the technical solution without departing from the inventive purpose of the invention made by inspired ordinary technicians in the art shall all fall within the protection scope of the invention.

1. An externally applied medicated wine for treating cervical spondylosis or lumbar spondylosis caused by hyperostosis, wherein it is made of the following raw materials in parts by weight: 10-15 parts of Arisaematis Rhizoma, 3-5.5 parts of Typhonium giganteum Engl., and 12-18 parts of baijiu (Chinese distilled spirits).

2. The externally applied medicated wine for treating cervical spondylosis or lumbar spondylosis caused by hyperostosis of claim 1, wherein it is made of the following raw materials in parts by weight: 10-15 parts of Arisaematis Rhizoma, 3-5 parts of Typhonium giganteum Engl., and 12-17 parts of baijiu.

3. The externally applied medicated wine for treating cervical spondylosis or lumbar spondylosis caused by hyperostosis of claim 2, wherein it is made of the following raw materials in parts by weight: 15 parts of Arisaematis Rhizoma, 5.5 parts of Typhonium giganteum Engl., and 18 parts of baijiu.

4. The externally applied medicated wine for treating cervical spondylosis or lumbar spondylosis caused by hyperostosis of claim 2, wherein it is made of the following raw materials in parts by weight: 10 parts of Arisaematis Rhizoma, 3 parts of Typhonium giganteum Engl., and 12 parts of baijiu.

5. The externally applied medicated wine for treating cervical spondylosis or lumbar spondylosis caused by hyperostosis of claim 1, wherein the Arisaematis Rhizoma is Arisaematis Rhizoma Recens, and the Typhonium giganteum Engl. is Typhonium giganteum Engl. Recens.

6. The externally applied medicated wine for treating cervical spondylosis or lumbar spondylosis caused by hyperostosis of claim 1, wherein the baijiu has an ABV above 50%.

7. An application of the externally applied medicated wine for treating cervical spondylosis or lumbar spondylosis caused by hyperostosis of claim 1 in preparing medications for hyperostosis.
